(19) United States
(12) Patent Application Publication
(10) Pub. No.: US 2008/0275301 A1
(43) Pub. Date: Nov. 6, 2008

(54) Sigmoidoscope With Integral Obturator

(75) Inventors: David Lubowski, New South Wales (AU); Peter Crispin Marsh, New South Wales (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Daltray Pty, Ltd, New South Wales (AU)

(21) Appl. No.: 11/579,963

(22) PCT Filed: May 10, 2004
(86) PCT No.: PCT/AU2004/000610

§ 371 Date: Nov. 19, 2007

(30) Foreign Application Priority Data
May 9, 2003 (AU) 2003902258

(51) Int. Cl. A61B 1/31

(52) U.S. Cl. 600/130; 600/138; 600/160

(57) ABSTRACT

A medical instrument used as a sigmoidoscope (1) for internal examination of the rectum and sigmoid colon of a patient. The instrument includes an outer tubular member in the form of a speculum (2) having a forward insertion end (3) and a rearward observation end (4). An inner tapered guide tube (10) is disposed at least partially within the speculum (2). A retractable obturation tube (15) is disposed between the speculum (2) and the inner guide tube (10) for relative movement between a retracted position substantially within the speculum (2) and an extended position in which the obturation tube (15) protrudes longitudinally beyond the forward end (3) of the speculum (2).

### FIELD OF THE INVENTION

This invention relates to medical instruments, and in particular to an instrument adapted for insertion into bodily cavities. The invention has been developed primarily as a sigmoidoscope for use in examination of the bowel cavity of a patient and for sigmoidoscopy generally. It will be appreciated, however, that the invention is not limited to this particular field of use.

### BACKGROUND OF THE INVENTION

The following description of the prior art is provided to place the invention in an appropriate technical context, and to enable the advantages flowing from it to be fully appreciated. References to the prior art should not, however, be interpreted as indicating that such art was well known, or formed part of the common general knowledge, in the relevant field at the priority date.

It is frequently necessary for specialised medical practitioners to be required to inspect the colon or rectum of a patient. This procedure is commonly conducted with a rigid sigmoidoscope, which in the past typically consisted of a metal tube or speculum adapted at one end for insertion into the rectum of a patient, and adapted at the other end for connection with a manifold. The manifold was provided with an observation window, and a rubber insufflation bulb connectable via a spigot to the manifold for fluid communication with the interior of the speculum whereby the bowel may be pressurised. An illumination system was also provided with which at least a portion of the interior of the bowel interior could be illuminated during examination. In the past, after use and before reuse on a subsequent patient, the metal tube was sterilised. Subsequently, sigmoidoscopes were designed such that components coming into direct contact with the patient were adapted to be disposed of after use to avoid the time and expense of cleaning and sterilising.

More recently, rigid sigmoidoscopes have been used which employ a disposable speculum in the form of a hollow, light transmissive, plastic tube. The disposable speculum is typically purchased in a clean or sterile condition sealed in a bag together with a disposable obturator adapted for use with the speculum. In use, the disposable speculum is coupled to a non-disposable fibre-optic head incorporating a hinged window through which an obturator or biopsy instrument may be inserted and removed coaxially through the interior of the speculum.

A light source is operatively coupled with the speculum via the fibre optic head so as to illuminate a circumferential end edge of the speculum via fibre-optics, whereby light is directed through the wall of the speculum into the anal canal or bowel cavity under examination. The fibre optic head is also provided with a spigot for connection, via a flexible tube, to an insufflation bulb with which the bowel may be insufflated with air. After initial insertion of the sigmoidoscope, the obturator is withdrawn through the speculum and discarded.

Upon conclusion of an examination, the inexpensive plastic speculum is disconnected from the fibre optic head and disposed of. The fibre-optic head together with the hinged window mounted thereto and the fibre-optic illumination means are relatively expensive components and are therefore not disposed of, but rather are retained for use with another speculum. The insufflation bulb may be disconnected between uses but commonly remains connected to the fibre optic head.

This practice gives rise to the potential for cross-infection from the reusable components of the sigmoidoscope, most notably the insufflation bulb and the fibre optic head. The present applicant recognised this potential, which led to the invention of a new form of sigmoidoscope, as described in patent application number PCT/AU99/00618, the full contents of which are hereby incorporated by cross-reference. That new form of sigmoidoscope addressed the problem of cross contamination via the insufflation bulb and the light head. However, a problem remained in relation to the obturator, which has not hitherto been addressed by the applicant's earlier invention, or the prior art.

In use, an obturator consisting essentially of an elongate stem terminating in a domed head is typically inserted axially through the interior of the speculum until the domed head protrudes beyond the remote end of the speculum, so as temporarily to close off the insertion end of the speculum. This facilitates initial insertion of the speculum, following which the contaminated obturator is withdrawn, and typically disposed of. This withdrawal is necessary in order to provide an unobstructed viewing path through the interior of the speculum. However, a fundamental problem with known sigmoidoscope designs is that as the obturator is withdrawn, it can and in practice almost inevitably does inadvertently come into contact with non-disposable parts of the sigmoidoscope, or with disposable parts that subsequently contact non-disposable parts. This creates significant potential for cross contamination.

A further problem is that when inserting the instrument or subsequently during the examination procedure, faecal matter may partially or completely occlude the open end of the speculum and hence obstruct the view. It is then necessary either to withdraw the instrument to clear the end, or reinsert the obturator or a cotton wool pledget to do so. This is inconvenient, time-consuming and gives rise to further potential for contamination of the instrument.

In an attempt to address these problems, the present applicant's earlier patent application referenced above discloses the use of an integral, disposable obturator in the form of a sleeve disposed slidably around the main tube of the speculum. At the insertion end, the obturator sleeve incorporates a plurality of resiliently deformable petals or tangs adapted in an extended configuration to curve inwardly, so as collectively to define a domed formation over the insertion end of the speculum. After insertion, the sleeve of the obturator is adapted to be telescopically withdrawn relative to the speculum. In this way, the petals or tangs resiliently retract around the speculum to a position behind the insertion end, whereby to provide an unobstructed viewing path through the interior of the speculum.

While addressing the problem of the potential for initial contamination from the obturator, this arrangement is not necessarily effective in clearing the insertion end of the speculum should it become occluded by faecal matter during a sigmoidoscopy procedure, and also presented other problems.

In the insertion configuration, longitudinal gaps or slots remain between the petals, bounded by the relatively sharp edges of the petals, giving rise to the possibility of pinching of the tissue of the patient. This in turn gives rise to the possibility of discomfort or even perforation of the bowel, with potentially serious consequences. Even in the retracted configuration, the edges of the petals remain exposed around the outer periphery the speculum, with the possibility of pinching and perforation of the colon wall, even after insertion.

Although these problems have been described in the context of sigmoidoscopy, it will be appreciated that similar difficulties may arise in analogous medical procedures.

It is an object of the present invention to provide an improved sigmoidoscope, which overcomes or ameliorates one or more of the disadvantages of the prior art, or at least provides a useful alternative.

### DISCLOSURE OF THE INVENTION

Accordingly, in a first aspect, the invention provides a medical instrument for internal examination of a patient, said instrument including an outer tubular member having a forward end and a rearward end, guide means disposed at least partially within the outer tubular member, and a retractable tubular member having a forward end and a rearward end, and being disposed generally between the outer tubular member and the guide means,

the retractable tubular member being relatively movable between a retracted position in which the forward end of the retractable tubular member is positioned substantially within the outer tubular member, and an extended position in which the forward end of the retractable tubular member protrudes longitudinally beyond the forward end of the outer tubular member.

Preferably, one or more of the outer tubular member, the guide means and the retractable tubular member include elements forming a drive mechanism such that relative movement effects a corresponding displacement of the retractable tubular member between the retracted and the extended positions.

Preferably, the guide means include an inner tubular member having a forward end and a rearward end, and being disposed generally within the outer tubular member such that the retractable tubular member is coaxially sandwiched in telescopic engagement between the inner and outer tubular members. Alternatively, the guide means may include locating formations projecting radially inwardly from the outer tubular member, for example.

In the preferred embodiment, the drive mechanism includes complementary engagement formations disposed respectively on the retractable tubular member and either or both of the inner and outer tubular members. In other embodiments, however, it will be appreciated that the drive mechanism could be formed from separate and discrete set of components.

In the preferred embodiment, the engagement formations include a spiral drive slot formed in the retractable tubular member, and at least one complementary drive boss extending radially outwardly from the inner tubular member for engagement with the drive slot, such that relative rotation between the inner and outer tubular members progressively effects a corresponding axial displacement of the retractable tubular member between the retracted and the extended positions. Preferably, the outer tubular member includes at least one complementary locating formation to prevent relative rotation between the retractable tubular member and the outer tubular member while the inner and outer members rotate relative to one another.

Alternatively, however, it will be appreciated that the drive mechanism may be configured such that movement of the retractable member may be effected by relative axial displacement between the inner and outer tubular members, or by some other form of actuation.

Preferably, the forward end of the retractable tubular member includes a plurality of generally longitudinally extending slits, defining between them a plurality of end segments adapted, in the extended configuration, to contract toward one another and thereby permit the retractable member to taper radially inwardly toward the forward end. Preferably, the end segments are curved radially inwardly along their respective lengths.

Preferably, in the extended position, the curved end segments of the retractable tubular member abut along respective adjacent longitudinal edges to form a substantially continuous domed structure, projecting smoothly beyond the forward end of the outer tubular member. Most preferably, the forward end of the outer tubular member tapers radially inwardly toward an open tip, the tapered end being configured to facilitate progressive contraction of the end segments toward one another into the domed configuration, as the retractable tubular member progressively moves through and beyond the forward end of the outer tubular member toward the extended position. The tapered end also holds these segments firmly in the domed configuration, once extended.

In the preferred embodiment, the medical instrument is adapted for use as a sigmoidoscope, wherein the rearward end of the outer tubular member constitutes an observation end and the forward end constitutes an insertion end of a speculum. Preferably also, the retractable tubular member constitutes an obturation tube, the forward end of which in the extended configuration constitutes an obturation formation, and in the retracted configuration provides a substantially unobstructed internal viewing path from the observation end through the insertion end of the speculum.

The forward end of the obturation tube is preferably configured such that in the extended configuration, the domed head provides a substantially closed, rounded tip protruding smoothly and generally continuously beyond the forward end of the speculum, thereby to provide an integral obturator adapted to facilitate initial insertion of the speculum, without requiring subsequent withdrawal and disposal of a discrete obturating element during the examination procedure.

Preferably, the forward end of the inner guide to tube includes an outer guide rim adapted to spread the end segments apart upon retraction of the obturation tube such that in the retracted position, the end segments are captively retained in an annular clearance space defined between the inner guide tube and the surrounding outer tube or speculum.

Preferably, the sigmoidoscope includes a closure element adapted to seal the observation end of the speculum, the closure element incorporating a substantially transparent observation window to permit visual inspection through the speculum from the observation end. The closure element may be releasably or fixedly attached.

In one preferred embodiment, the sigmoidoscope includes at least one reusable part, at least one disposable part, and manually operable insufflation means, the speculum being adapted for insertion into a bowel cavity of a patient, and the insufflation means being adapted in use to insufflate the bowel cavity through the speculum with an insufflation medium to facilitate a visual examination from the observation end through the closure element, the insufflation medium being thereby susceptible to contamination from within the bowel cavity, and the sigmoidoscope further including contamination prevention means operable such that no reusable part is exposed to any contaminated insufflation medium or faecal matter during the examination.

A “contaminant” as herein defined is any agent capable of infection and includes, without limitation, viruses, bacteria, fungi, protozoa, mycoplasma and organic or inorganic carriers of any of the above. “Contamination” carries the same sense of meaning.

The contamination prevention means may include, for example, a non-return valve, a filter such as a nanopore filter, an electrostatic precipitator or other means adapted to prevent internal surfaces of the reusable components from becoming contaminated either by contact with airborne contaminants via the insufflation medium, or by direct contact with a patient or other contaminated parts of the instrument. In this context, it will be understood that the term “insufflation means” includes any tubing, fluid conduits or other components communicating between an insufflation bulb or other source of insufflation medium, and the sigmoidoscope.

The sigmoidoscope preferably also includes an optical head incorporating an eyepiece and illumination means to facilitate visual examination through the speculum via the observation window in the closure element. Preferably, the speculum is formed from an optically transparent or translucent material adapted to conduct light from the illumination means at the observation end through to the insertion end of the speculum, thereby to illuminate the bowel cavity during the examination procedure.

In one particularly preferred embodiment, the speculum includes a side wall extending along the tube from the observation end to the insertion end to define a lumen, and connection means to allow a reusable light source to be connected to the observation end to project light through the speculum into the rectum and sigmoid colon of patient. Preferably, the instrument includes manually operable insufflation means, adapted to insufflate the colon of the patient through the speculum with an insufflation medium susceptible to contamination from within the colon. In this embodiment, the insufflation medium is preferably conveyed directly from the insufflation means to the lumen by a gas conveying tube.

Preferably, the instrument further includes a gas-tight observation window disposed to prevent passage of the insufflation medium from the lumen of the speculum to the connection means for the light source. Preferably also, the instrument includes contamination prevention means, effectively insulating the reusable light source from exposure to any contaminated insufflation medium.

The insufflation means preferably include an inflation bulb and the gas conveying insufflation tube is preferably adapted for connection to a hollow spigot extending outwardly from the inner tubular member or guide tube, the insufflation tube and spigot being thereby adapted to establish fluid communication between the insufflation bulb and the interior region of the sigmoidoscope. In this case, the insufflation medium is preferably air.

In the preferred embodiment, the speculum, obturation tube, inner guide tube, closure element and insufflation means are effectively disposable, while the optical head and illumination means are readily re-usable, in the conventional sense that these terms are generally understood by those skilled in the art of medical and surgical practice.

### BRIEF DESCRIPTION OF THE DRAWINGS

A preferred embodiment of the invention will now be described, by way of example only, with reference to the accompanying drawings in which:—

FIG. 1 is a perspective view showing a medical instrument, adapted for use as a sigmoidoscope, incorporating an insufflation bulb, optical head and light source, according to a first embodiment of the invention;

FIG. 2 is an exploded perspective view of the sigmoidoscope of FIG. 1 showing the primary constituent components including the inner guide tube, obturation tube, outer speculum and closure element;

FIG. 3 is a side elevation view of the sigmoidoscope shown in FIGS. 1 and 2;

FIG. 4 is an end elevation showing the observation end of the sigmoidoscope of FIG. 3;

FIG. 5 is an end elevation showing the insertion end of the sigmoidoscope of FIG. 3;

FIG. 6 is an enlarged perspective view showing the inner guide tube of the sigmoidoscope;

FIG. 7 is an enlarged perspective view of the outer tube or speculum of the sigmoidoscope;

FIG. 8 is an enlarged perspective view of the retractable obturation tube;

FIG. 9 is an enlarged perspective view of the closure element;

FIG. 10 is an enlarged perspective view showing the retractable obturation tube and end cap operatively mounted on the inner guide tube as a sub-assembly;

FIG. 11 is a cross-sectional side elevation, showing the obturation tube in a fully retracted configuration;

FIG. 12 is a cross-sectional side elevation similar to FIG. 11, showing the obturation tube in a partially retracted configuration; and

FIG. 13 is a cross-sectional side elevation showing the sigmoidoscope with the obturation tube in the fully extended configuration.

### PREFERRED EMBODIMENTS OF THE INVENTION

Referring to the drawings, the invention provides a medical instrument adapted for use as a sigmoidoscope 1. The sigmoidoscope includes an outer tubular member in the form of a speculum 2, having a forward insertion end 3 and a rearward observation end 4. An inner tubular member in the form of tapered inner guide tube 10 incorporates a forward end 11 including an outer guide rim 12 and a rearward end 13. The guide tube is disposed substantially within the speculum 2, with the forward end 11 recessed well behind the forward end 3 of the speculum.

The sigmoidoscope 1 further includes a retractable tubular member in the form of obturation tube 15, having a forward obturation end 16 and a rearward driven end 17. The obturation tube 15 is movable between a retracted configuration in which the forward end 16 is positioned substantially within the speculum as shown in FIG. 11, and an extended configuration in which the forward end protrudes longitudinally beyond the forward end of the speculum, as shown in the 13.

As best seen in FIGS. 11 to 13, the rearward end of the inner guide tube incorporates an outwardly depending circumferential locating flange 20 adapted for sealing engagement with a corresponding inwardly depending circumferential shoulder flange 21 formed on the rearward end of the speculum. These interlocking flanges permit relative rotation between the inner and outer tubes, but prevent relative axial displacement during normal operation. The flanges can be disengaged, however, to permit disassembly.

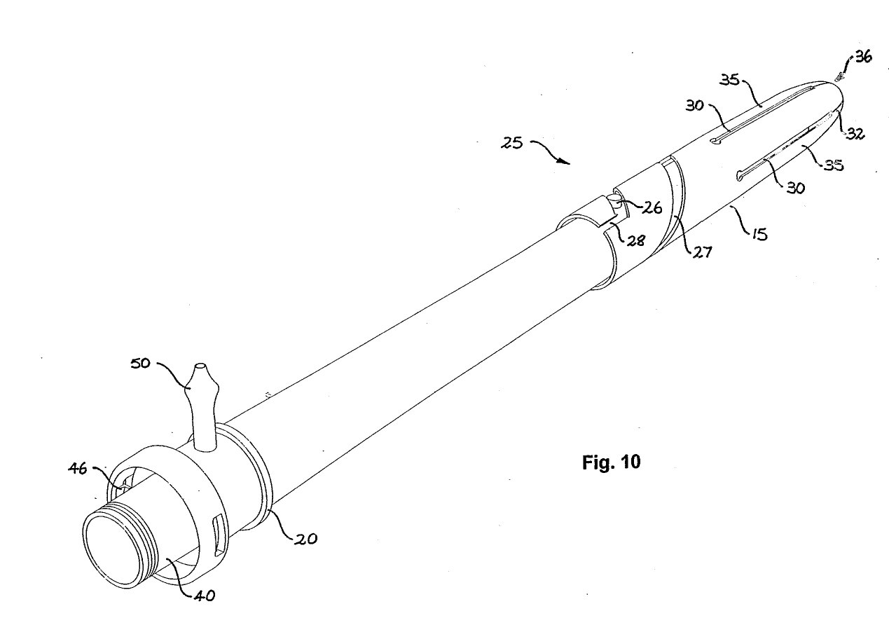

As best seen in FIG. 10, the instrument further includes a drive mechanism 25, comprising a pair of diametrically opposed drive bosses 26 extending radially outwardly from the inner guide tube, and a corresponding pair of spiral drive slots 27 formed in the sidewall toward the rear driven end of the retractable obturation tube 15. A straight axial entry section 28 to one of the drive slots 27 facilitates assembly and disassembly of these two components.

The forward insertion end of the obturation tube 15 includes a plurality of longitudinally extending locating slots 30 adapted respectively for engagement by a corresponding plurality of complementary axial locating ribs 31 extending radially inwardly from the forward end of the surrounding speculum, as best seen in FIGS. 11 to 13. Engagement of these locating ribs with the corresponding slots allows a limited degree of relative axial displacement between the obturation tube and the speculum, while preventing relative rotation. In this way, it will be appreciated that relative rotation between the inner guide tube and the outer speculum progressively effects a corresponding axial displacement of the obturation tube between the retracted and extended positions.

The forward end of each locating slot 30 terminates in a longitudinal slit 32, which continues through to the tip to divide the forward end of the obturation tube into a corresponding series of elongate end segments 35. The end segments are curved inwardly toward one another along their respective lengths, such that in the extended configuration, they abut along their respective longitudinal edges as defined by the slits 32, so as collectively to form a substantially smooth and effectively continuous domed structure 36.

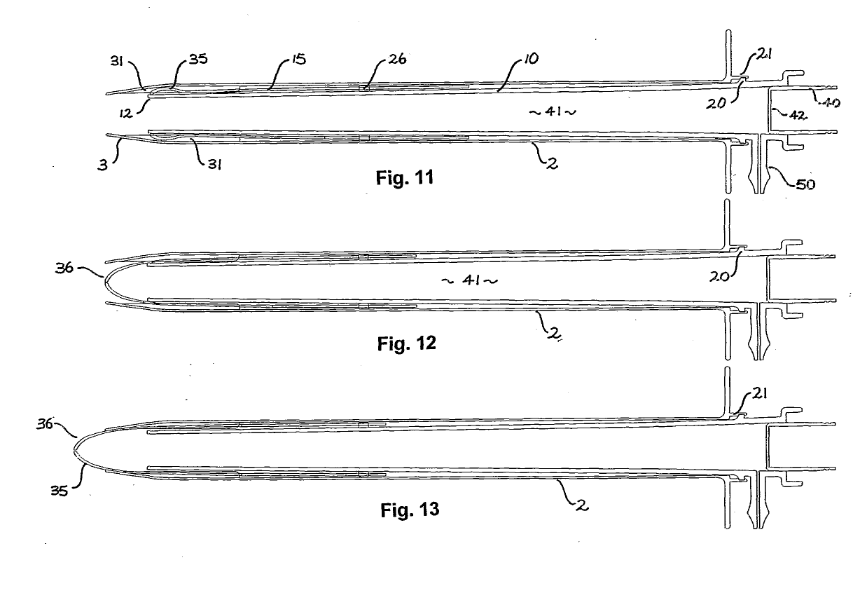

The forward end of the speculum tapers radially inwardly toward the insertion end. This taper assists the progressive contraction of the end segments of the obturation tube toward one another and into the domed configuration as the obturation tube moves progressively through and beyond the forward end of the speculum, toward the extended position, as shown in FIG. 13. The tapered end of the speculum then holds the domed formation tightly together in the extended position. As the obturation tube is retracted into the speculum, the end segments 35 are progressively and resiliently spread apart by the outer guide rim 12 on the forward end of the inner guide tube. In this retracted configuration, as best shown in FIG. 11, the end segments 35 of the obturation tube are captively located in the annular clearance space defined between the inner guide tube and the surrounding speculum, to provide an unobstructed axial viewing path through the sigmoidoscope. Importantly, in the intermediate configuration as shown in FIG. 12, the end segments 35 of the obturation tube come together in the closed configuration, which is their natural rest position, to form the effectively continuous domed structure as previously described, substantially within the surrounding speculum.

The instrument further includes a closure element in the form of end cap 40 adapted for insertion into the observation end of the inner guide tube, which protrudes rearwardly beyond the surrounding speculum, with an interference fit. This fit must be sufficiently tight to seal the interior region 41 of the sigmoidoscope at the observation end and prevent inadvertent ejection of the end cap under insufflation pressure. The face of the end cap perpendicular to the longitudinal axis of the instrument incorporates a substantially transparent observation window 42. For simplicity and efficiency of production, the entire end cap may ideally be formed from a transparent plastics material.

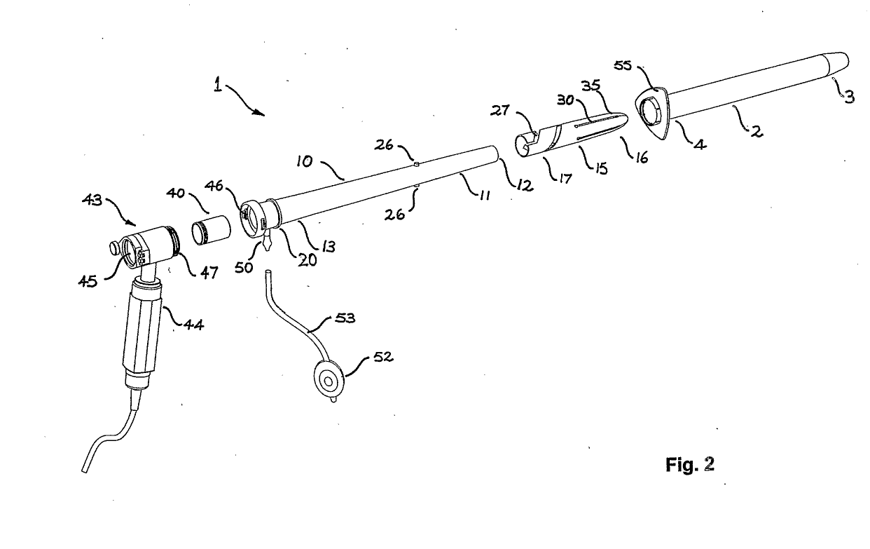

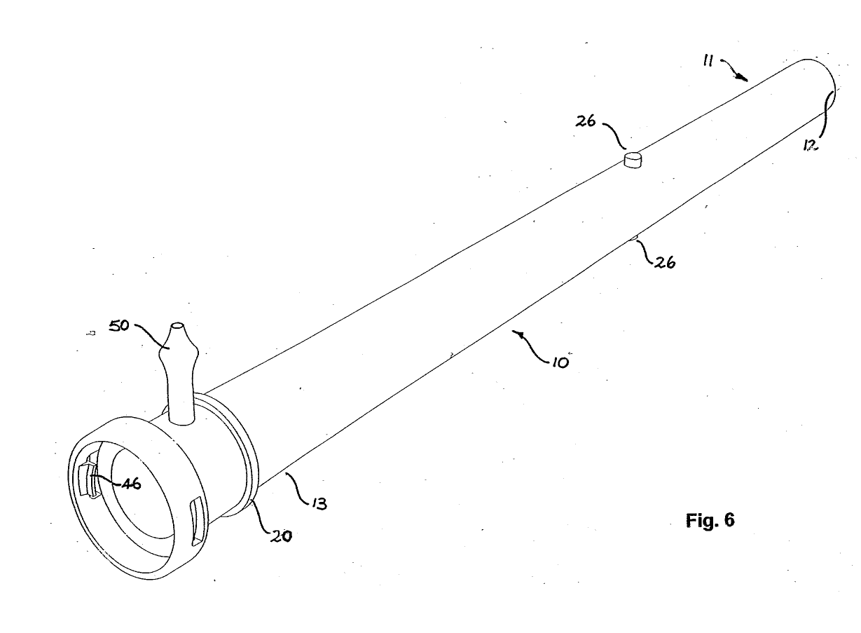

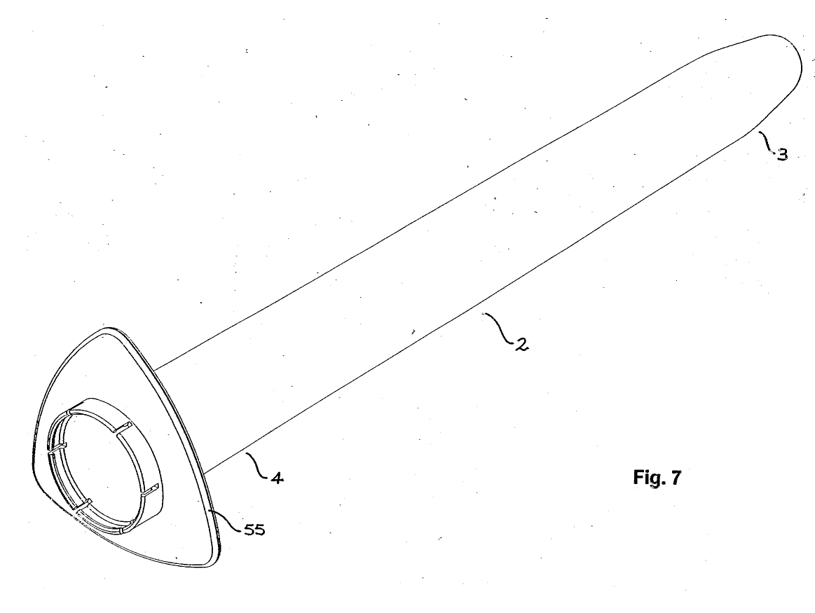

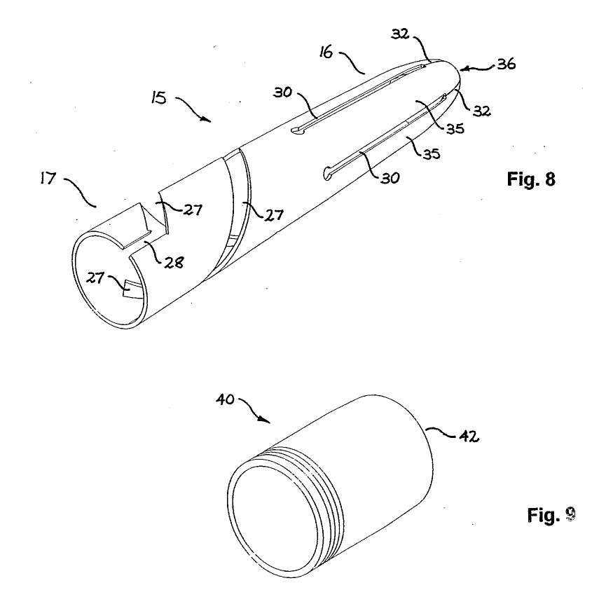

An optical head 43 incorporating a light source 44 and a hinged eyepiece 45 is adapted for mounting over the end cap on the observation end of the inner guide tube. This mounting is achieved by means of inwardly depending pitched screw lugs 46 on the proximal end of the guide tube, as best seen in FIG. 6, which are adapted for engagement with a correspondingly pitched thread 47 formed on the outer forward end of the optical head, as best seen in FIG. 2. Alternatively, a bayonet or other suitable type of selectively releasable fitting may be used.

The rearward end of the inner guide tube also incorporates a tubular radial spigot 50 adapted for connection to a manual insufflation bulb 52 by means of a flexible insufflation conduit 53, to establish fluid communication between the insufflation bulb and the interior region 41 of the speculum. The speculum tube further includes a large radial flange 55 adapted to facilitate manipulation and to prevent over-insertion of the instrument.

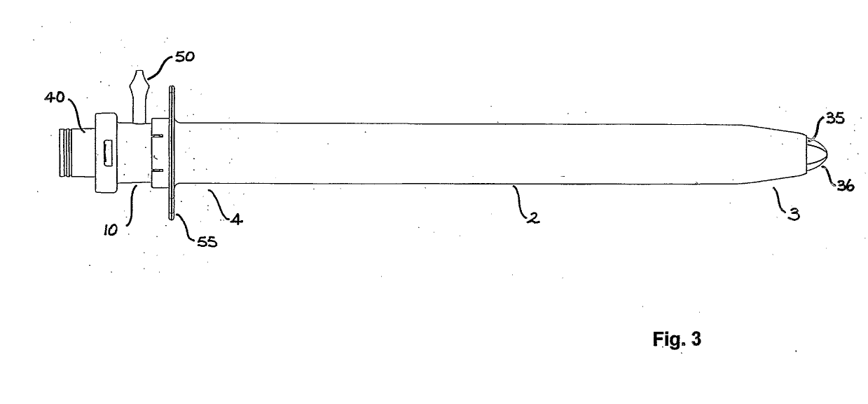

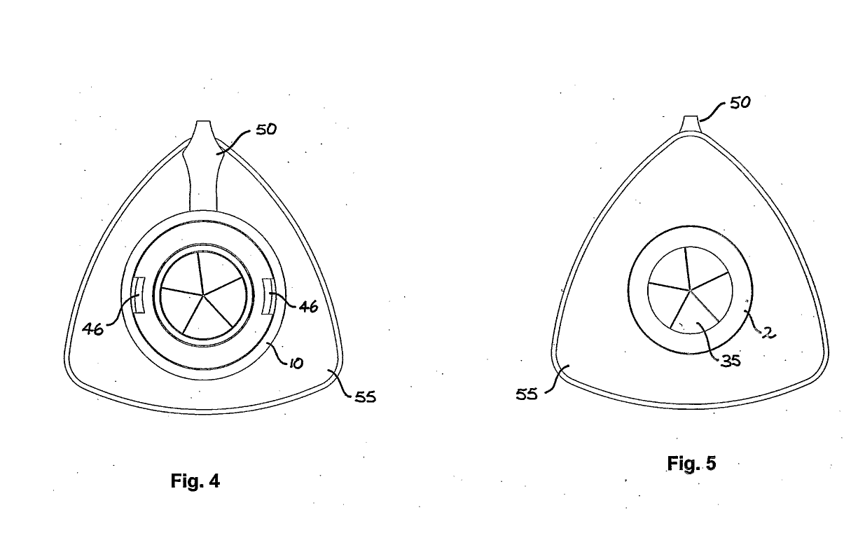

Turning now to describe the operation of the instrument, the sigmoidoscope is initially assembled to the stage shown in FIG. 3. The insufflation bulb and end cap are then attached, but the optical head is not yet fitted. The outer tube or speculum is manually rotated in a clockwise direction relative to the inner guide tube, such that the drive mechanism displaces the obturation tube forwardly into the extended position. In this position, the elongate end segments of the obturation tube are urged together into the closed configuration by virtue of their own resiliency to form the effectively continuous domed head 36 which protrudes smoothly beyond the forward end of the spectrum. The end segments are securely retained in that configuration by the inwardly tapered end 3 of the surrounding speculum. The domed head thereby effectively forms an integral obturator, as best shown in FIGS. 3 and 13.

With the instrument thus assembled and the domed head of the obturation tube protruding smoothly beyond the forward end of the speculum, the sigmoidoscope is suitably lubricated and inserted into the bowel cavity of the patient, with the integral obturator greatly facilitating the rectal insertion procedure.

Once the instrument is in position, the inner guide tube is manually rotated relative to the outer speculum in the reverse direction, whereby the drive mechanism progressively retracts the obturation tube. During the retraction process, the end segments 35 are progressively and resiliently spread apart by the outer rim 12 of the guide tube, and withdrawn into the annular clearance space between the inner guide tube and the surrounding speculum, as shown in FIG. 11. This provides an unobstructed axial viewing path through the instrument. Importantly, during this process, the obturator does not need to be physically removed from the speculum and so the possibility of contamination of the rearward end of the instrument by this mechanism is substantially eliminated.

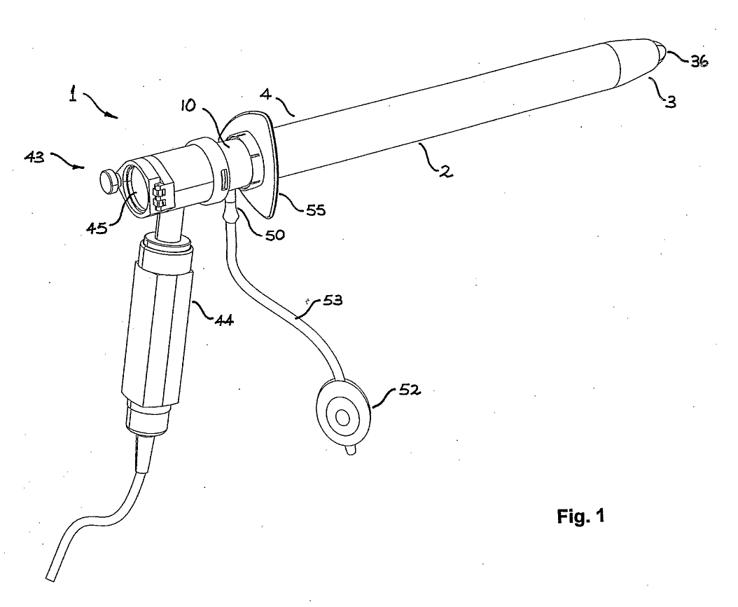

Next, the optical head is screwed on and secured to the observation end of the inner guide tube over the end cap, as best seen in FIGS. 1 and 2. The patient's bowel is then insufflated by manual squeezing of the insufflation bulb to facilitate clearer visual inspection.

The light source in the optical head is then energised, whereby light from the source travels along the speculum, which is formed from an optically translucent material, to the insertion end, from where it is emitted from the tube. This illuminates the insufflated area of the bowel in the region observable by the physician looking through the eyepiece.

If the insertion end of the speculum should become occluded by faecal matter at any stage during the procedure, the occlusion may be readily removed by relative rotation of the inner and outer tubes, in the manner previously described. This causes the obturation tube initially to extend into the intermediate position with the end segments in the closed configuration substantially within and behind the forward end of the speculum, as shown in FIG. 12. Further rotation moves the obturation tube into the fully extended position, protruding beyond the forward end of the speculum, as shown in FIG. 13. By this means, the domed obturation formation acts to physically clear any occlusion or obstruction from the speculum. The inner and outer tubes are then rotated in the reverse direction, to withdraw the obturation tube once again into the retracted position, as shown in FIG. 11. Importantly, this process can be effected without temporary removal of the optical head and end cap, and without insertion and subsequent withdrawal of a separate obturator or pledget, as would be required using known sigmoidoscopes. This substantially eliminates another mechanism for potential contamination.

After the visual inspection has been completed, the optical head is detached, and the speculum removed from the patient. The speculum, inner guide tube and obturation tube can then be discarded as a disposable assembly. It is envisaged that the insufflation bulb and tube would also be disposed of with the speculum, since all of these components would be potentially contaminated as a result of direct contact with the patient, and are readily replaceable. With appropriate bacterial filtration mechanisms or sterilisation protocols, however, the insufflator in some embodiments of the invention may be reused.

The optical head and light source, which will not have come into direct contact with the patient, are retained for use in with new sigmoidoscopes on subsequent patients. This is important because the capital cost of these precision instruments is too high to justify disposal after each use, and the cleaning and decontamination processes that would otherwise be required using known sigmoidoscopy techniques is time-consuming and expensive.

The invention thus provides a simple, elegant, efficient and cost-effective medical instrument, particularly well adapted for use as a sigmoidoscope, in which the components susceptible to contamination are inexpensive and readily disposable, while the more costly components are effectively isolated from contamination so as to be readily reusable. In particular, by providing an integral obturator that does not require removal as a discrete component following initial insertion of the instrument, a potential contamination mechanism has been entirely eliminated. Furthermore, by providing a system for clearing occlusions during the procedure without having to open the speculum, another potential contamination mechanism has been substantially eliminated. In all these and other respects, the invention represents both a practical and commercially significant improvement over the prior art.

Although the invention has been described with reference to specific examples, it will be appreciated by those killed in the art that the invention may be embodied in many other forms.

1-25. (canceled)

26. A medical instrument for internal examination of a patient, said instrument including an outer tubular member having a forward end and a rearward end, guide means disposed at least partially within the outer tubular member, and a retractable tubular member having a forward end, a rearward end and being disposed generally between the outer tubular member and the guide means,
the retractable tubular member being relatively movable between a retracted position in which the forward end of the retractable tubular member is positioned substantially within the outer tubular member, and an extended position in which the forward end of the retractable tubular member protrudes longitudinally beyond the forward end of the outer tubular member,
the forward end of the retractable tubular member including a plurality of generally longitudinally extending slits, defining between them a plurality of inwardly curved end segments,
the end segments being adapted, in the extended configuration, to contract toward one another and substantially abut along respective adjacent longitudinal edges, so as to form a domed obturation structure projecting beyond the forward end of the outer tubular member.

27. An instrument according to claim 26 herein one or more of the outer tubular member, the guide means and the retractable tubular member include elements forming a drive mechanism such that relative movement effects a corresponding displacement of the retractable tubular member between the retracted and the extended positions.

28. An instrument according to claim 26, wherein the guide means include an inner tubular guide member having a forward end and a rearward end, the inner tubular guide member being disposed generally within the outer tubular member such that the retractable tubular member is coaxially sandwiched in telescopic engagement between the inner and outer tubular members.

29. An instrument according to claim 28, wherein the drive mechanism includes complementary engagement formations disposed respectively on the retractable tubular member and either or both of the inner and outer tubular members.

30. An instrument according to claim 29, wherein said engagement formations include a spiral drive slot formed in the retractable tubular member, and at least one complementary drive boss extending radially outwardly from the inner tubular member for engagement with the drive slot, such that relative rotation between the inner and outer tubular members progressively effects a corresponding axial displacement of the retractable tubular member between the retracted and the extended positions.

31. An instrument according to claim 30, wherein the outer tubular member includes at least one complementary locating formation to prevent relative rotation between the retractable tubular member and the outer tubular member while the inner and outer members rotate relative to one another.

32. An instrument according to claim 28, wherein the drive mechanism is configured such that movement of the retractable member is effected by relative axial displacement between the inner and outer tubular members.

33. An instrument according to claim 26, wherein the forward end of the outer tubular member tapers radially inwardly toward an open tip, the tapered forward end being thereby configured to facilitate progressive contraction of the end segments toward one another into the domed configuration, as the retractable tubular member progressively moves through and beyond the forward end of the outer tubular member toward the extended position.

34. An instrument according to claim 26, wherein the tapered forward end is adapted to retain the segments firmly in the domed configuration, when the retractable tubular member is in the extended position.

35. An instrument according to claim 26, being adapted for use as a sigmoidoscope, wherein the rearward end of the outer tubular member constitutes an observation end, and the forward end constitutes an insertion end of a speculum.

36. An instrument according to claim 28, wherein the retractable tubular member-constitutes an obturation tube, the forward end of which in the extended configuration constitutes an obturation formation, and in the retracted configuration provides a substantially unobstructed internal viewing path from the observation end through the insertion end of the speculum.

37. An instrument according to claim 29, wherein the forward end of the obturation tube is configured such that in the extended configuration, the domed head provides a substantially closed, rounded tip protruding smoothly and generally continuously beyond the forward end of the speculum, thereby to provide an integral obturator adapted to facilitate initial insertion of the speculum, without requiring subsequent withdrawal and disposal of a discrete obturating element during examination of the rectum and sigmoid colon of a patient.

38. An instrument according to claim 30, wherein the forward end of the inner guide tube includes an outer guide rim adapted progressively to spread the end segments apart upon retraction of the obturation tube such that in the retracted position, the end segments are captively retained in an annular clearance space defined between the inner guide tube and the surrounding outer tube or speculum.

39. An instrument according to any one of claim 28, further including a closure element adapted to seal the observation end of the speculum, the closure element incorporating a substantially transparent observation window to permit visual inspection through the speculum from the observation end.

40. An instrument according to claim 39, wherein the closure element is releasably attached.

41. An instrument according to 26, including at least one reusable part, at least one disposable part, and manually operable insufflation means, the outer tubular member constituting a speculum adapted for insertion into a bowel cavity of a patient, and the insufflation means being adapted in use to insufflate the bowel cavity through the speculum with an insufflation medium to facilitate a visual examination from the observation end through a closure element, the insufflation medium being thereby susceptible to contamination from within the bowel cavity, and the sigmoidoscope further including contamination prevention means operable such that no reusable part is exposed to any contaminated insufflation medium or faecal matter during the examination.

42. An instrument according to claim 26, being adapted for use as a sigmoidoscope, wherein the outer tubular member constitutes a disposable speculum comprising:
an elongate substantially rigid tube having an observation end and an insertion end adapted for insertion into the rectum and sigmoid colon of a patient;
a side wall extending along the tube from the observation end to the insertion end to define a lumen;
connection means adjacent the observation end of the speculum to allow a reusable light source to be connected to the observation end and to project light through the speculum into the rectum and sigmoid colon of patient;
and manually operable insufflation means adapted to insufflate the colon of the patient through the speculum with an insufflation medium susceptible to contamination from within the colon, the insufflation medium being conveyed directly from the insufflation means to the lumen by a gas conveying tube.

43. An instrument according to claim 26, further including a gas-tight observation window disposed to prevent passage of the insufflation medium from the lumen of the speculum to the connection means for the light source.

44. An instrument according to claim 41, further including contamination prevention means, effectively insulating the reusable light source from exposure to any contaminated insufflation medium.

45. An instrument according to claim 42, wherein the speculum is formed from an optically transparent or translucent material adapted to conduct light from the light source at the observation end through to the insertion end of the speculum, thereby to illuminate the colon during the examination procedure.

46. An instrument according to claim 42, wherein the insufflation means include an inflation bulb and a flexible insufflation tube adapted for connection to a hollow spigot extending outwardly from the inner tubular member, the insufflation tube and spigot being thereby adapted to establish fluid communication between the insufflation bulb and the interior region of the sigmoidoscope.

47. An instrument according to claim 42, wherein the speculum, obturation tube, inner guide tube, closure element and insufflation means are disposable, and wherein the light source is reusable.

48. A medical instrument for internal examination of a patient, said instrument including an outer tubular member having a forward end and a rearward end, guide means disposed at least partially within the outer tubular member, and a retractable tubular member having a forward end, a rearward end and being disposed generally between the outer tubular member and the guide means,
the retractable tubular member being relatively movable between a retracted position in which the forward end of the retractable tubular member is positioned substantially within the outer tubular member, and an extended position in which the forward end of the retractable tubular member protrudes longitudinally beyond the forward end of the outer tubular member.

49. A medical instrument according to claim 48, wherein the forward end of the retractable tubular member includes a plurality of generally longitudinally extending slits, defining between them a plurality of inwardly curved end segments.

50. A medical instrument according to claim 49, wherein the end segments are adapted, in the extended configuration, to contract toward one another and substantially abut along respective adjacent longitudinal edges, so as to form a domed obturation structure projecting beyond the forward end of the outer tubular member.
